(19) United States
(12) Patent Application Publication
(10) Pub. No.: US 2006/0233842 A1
(43) Pub. Date: Oct. 19, 2006

(54) Microemulsion composition for oral administration of biphenyldimethyldicarboxylate

(76) Inventors: Jong-Soo Wood, Suwon-si, Kyungki-do (DO); Won-Tae Jung, Seoul (KR); Ae-Guk Kim, Incheon (KR); Tae-Sook Yoo, Seoul (KR); Moon-Soo Kim, Namyangju-si, Kyungki-do (KR); Min-Sik Hwang, Yongin-si, Kyungki-do (KR)

Correspondence Address: SUGHRUE MION, PLLC, 2100 PENNSYLVANIA AVENUE, N.W., SUITE 800, WASHINGTON, DC, 20037, US

(21) Appl. No.: 10/536,351

(22) PCT Filed: Nov. 29, 2003
(86) PCT No.: PCT/KR03/02610

§ 371 Date: Apr. 4, 2006

(30) Foreign Application Priority Data
Nov. 29, 2002 (KR) 10-2002-0075126

(51) Int. Cl. A61K 9/00

(52) U.S. Cl. 424/400

(57) ABSTRACT

A microemulsion composition comprising biphenyldimethyldicarboxylate (DDB), a co-surfactant, a surfactant and an oil provides an improved stability and a high in vivo bioavailability of biphenyldimethyldicarboxylate when orally administered.

### FIELD OF THE INVENTION

The present invention relates to an improved microemulsion composition for oral administration of biphenyldimethyldicarboxylate (DDB).

### BACKGROUND OF THE INVENTION

Biphenyldimethyldicarboxylate, a synthetic derivative of Schizandrin C which is one of the active ingredients isolated from Schizandra chinensis, is known to be useful for treating liver disease including acute/chronic hepatitis caused by virus, chronic liver disease and liver impairment by drug toxicity, by lowering SGPT (serum glutamic pyruvic transaminase).

However, the bioavailability of orally administered biphenyldimethyldicarboxylate is unsatisfactorily low due to its low solubility in water (about 3.6 μg/ml at 25° C. water), and accordingly, there have been reported a number of methods to improve the solubility thereof.

For example, Korean Patent No. 10-154612 discloses a method for preparing a biphenyldimethyldicarboxylate solid dispersion using Poloxamer. However, the manufacturing process of the dispersion is very complicated and the in vivo bioavailability of biphenyldimethyldicarboxylate thereof is still limited.

Further, Korean Patent No. 10-201907 discloses a soft capsule of biphenyldimethyldicarboxylate comprising polyethyleneglycol as a solvent. However, this preparation fails to provide a desired absorption rate due to the precipitation of the biphenyldimethyldicarboxylate upon contact with the aqueous body fluid.

On the other hand, Korean Patent No. 10-306736 discloses a microemulsion comprising triacetine as a solvent to overcome the above problems. However, triacetine is toxic, LD₅₀ for acute oral toxicity being 1.1 g/kg (Handbook of pharmaceutical excipients, p570˜571, 3^{rd} Ed., American pharmaceutical association, Washington D.C.).

### SUMMARY OF THE INVENTION

Accordingly, it is an object of the present invention to provide a microemulsion composition for oral administration of biphenyldimethyldicarboxylate having improved bioavailability.

### BRIEF DESCRIPTION OF THE DRAWINGS

The above and other objects and features of the present invention will become apparent from the following description of the invention, when taken in conjunction with the accompanying drawings, which respectively show:

FIGS. 1a to 1c: the dissolution rates of the inventive biphenyldimethyldicarboxylate (DDB) preparation of Examples 1, 3 and 10, and a commercially available biphenyldimethyldicarboxylate preparation (Nissel® tab.), respectively, in distilled water (FIG. 1a: Example 1, FIG. 1b: Example 3, FIG. 1c: Example 10);

FIGS. 2 to 4: the dissolution rates of the inventive biphenyldimethyldicarboxylate (DDB) preparation of Example 1 and a commercially available biphenyldimethyldicarboxylate preparation (Nissel® tab.), respectively, in buffers (FIG. 2: pH 1.2 buffer, FIG. 3: pH 4.0 buffer, FIG. 4: pH 6.8 buffer);

FIG. 5: the particle size distribution of the emulsified microparticles formed from the inventive biphenyldimethyldicarboxylate preparation of Example 1 upon contact with an aqueous solution; and

FIG. 6: the bioavailabilities of the inventive biphenyldimethyldicarboxylate preparation of Example 1 and a commercially available biphenyldimethyldicarboxylate preparation (Nissel® tab.).

### DETAILED DESCRIPTION OF THE INVENTION

In accordance with one aspect of the present invention, there is provided a microemulsion composition for oral administration of biphenyldimethyldicarboxylate (DDB) comprising biphenyldimethyldicarboxylate, a co-surfactant, a surfactant and an oil.

The respective components employed for the preparation of the inventive microemulsion composition are described in detail as follows.

(1) Active Ingredient

In the present invention, water-insoluble biphenyldimethyldicarboxylate is used as an active ingredient.

(2) Co-Surfactant

In the present invention, the co-surfactant serves to dissolve the water-insoluble biphenyldimethyldicarboxylate (active ingredient) and to emulsify the preparation. Representative examples thereof include a non-toxic transcutol (diethyleneglycol monoethylether), polyethyleneglycol (preferably having a molecular weight of 200 to 600) or a mixture thereof.

The LD₅₀ for acute oral toxicity of transcutol is 7.95 ml (specific gravity, 0.989)/kg (Gattefosse product profile), and that of polyethyleneglycol is 28.9 g/kg (Handbook of pharmaceutical excipients, p570˜571, 3^{rd} Ed., American pharmaceutical association, Washington D.C.). Accordingly, the above co-surfactants are much safer to patients than triacetine (which has LD₅₀ for acute oral toxicity of 1.1 g/kg).

(3) Surfactant

The surfactant used in the present invention may be any one of the pharmaceutically acceptable surfactants, which can be used to form a stable emulsion of oils and hydrophilic ingredients such as the co-surfactant in water. Representative examples of the surfactant include:

\textcircled{1} polyoxyethylene glycolated natural or hydrogenated vegetable oils such as polyoxyethylene glycolated natural or hydrogenated castor oil (Cremophor®, BASF; and HCO®, Nikkol),

\textcircled{2} polyoxyethylene-sorbitan-fatty acid esters wherein fatty acid is mono- or tri-lauric, palmitic, stearic or oleic acid (Tween®, ICI),

\textcircled{3} polyoxyethylene fatty acid esters such as polyoxyethylene stearic acid ester (Myrj®, ICI),

\textcircled{4} polyoxyethylene-polyoxypropylene block copolymer (Poloxamer®, Pluronic® or Lutrol®, BASF),

\textcircled{5} mono-, di- or mono/di-glycerides such as caprylic/capric acid mono- and di-glycerides Imwitor®, Hûls),

\textcircled{6} sorbitan fatty acid esters such as sorbitan monolauryl, sorbitan monopalmityl and sorbitan monostearyl esters (Span®, ICI), and

\textcircled{7} trans-esterification products of natural vegetable oil triglycerides and polyalkylene polyols (Labrafil® and Labrasol®, Gattefosse) etc.

The above-mentioned surfactants can be used separately or as a mixture, and polyoxyethylene glycolated hydrogenated vegetable oils are preferred.

(4) Oil

The oil may be any one of the pharmaceutically acceptable oils which is compatible with the surfactant and stably emulsified in water to form a stable microemulsion. Representative examples of the oil include:

\textcircled{1} fatty acid triglycerides, preferably medium chain fatty acid triglycerides, such as fractionated coconut oil (Miglyol® 812N, Hûls; Captex®, Abitec),

\textcircled{2} mono-, di- or mono/di-glycerides, preferably mono- or di-glycerides of oleic acid,

\textcircled{3} esters of fatty acids and monovalent alkanols, preferably esters of C₈₋₂₀ fatty acids and C₂₋₃ monovalent alkanols, such as isopropyl myristate, isopropyl palmitate, ethyl linoleate and ethyl oleate,

\textcircled{4} propyleneglycol mono- or di-fatty acid esters such as propyleneglycol dicaprylate, propyleneglycol monocaprylate, propyleneglycol dilaurate, propyleneglycol isostearate, propyleneglycol monolaurate and propyleneglycol ricinolate,

\textcircled{5} carbohydrates such as squalene and squalane, and

\textcircled{6} tocopherols such as tocopherol, tocopherol acetate, tocopherol succinate and polyethyleneglycol-1000-tocopherol succinate (TPGS).

The above-mentioned oils can be used separately or as a mixture, and medium chain fatty acid triglycerides and propyleneglycol monocaprylate are more preferable.

In the preparation of the inventive microemulsion composition, the active ingredient (biphenyldimethyldicarboxylate), the co-surfactant, the surfactant and the oil may be used in amounts corresponding to a weight ratio in the range of 1:5˜300:1˜300:1˜300, preferably, 1:30˜200:40˜200:35˜200.

In addition, the inventive composition may comprise pharmaceutically acceptable additives for oral administration, e.g., viscosity controlling agents, aromatics, anti-oxidants or preservatives etc.

The inventive composition may be prepared by mixing and dissolving said components uniformly, and it forms emulsified microparticles having an average diameter of below 300 nm on contacting an aqueous medium.

The microemulsion composition of the present invention may be formulated into a soft or hard capsule, in accordance with any of the conventional procedures.

A typical daily dose of biphenyldimethyldicarboxylate ranges from about 75 to 150 mg, and can be administered in a single dose or in divided doses.

The following Examples are intended to further illustrate the present invention without limiting its scope.

### EXAMPLE 1

### Preparation of a Soft Capsule Containing a Microemulsion Composition

A soft capsule was prepared using the following ingredients:

| | Quantity(mg/capsule) |
|---|---|
| Biphenyldimethyldicarboxylate | 3 |
| Transcutol | 160 |
| Polyethyleneglycol 400 | 40 |
| Cremophor ® RH40 (BASF) | 136 |
| Propyleneglycol monocaprylate (NIKKOL) | 72 |
| Captex ® 300 (Abitec) | 72 |

Biphenyldimethyldicarboxylate was dissolved in a mixture composed of transcutol and polyethyleneglycol 400, and other ingredients were added thereto and dissolved to obtain a microemulsion pre-concentrate. Then, the resulting pre-concentrate was filled into a soft capsule in accordance with the conventional method described in the General Preparation Rule of the Korean Pharmacopoeia.

### EXAMPLE 2

### Preparation of a Soft Capsule Containing a Microemulsion Composition

A soft capsule was prepared by the procedure of Example 1 using the following ingredients:

| | Quantity(mg/capsule) |
|---|---|
| Biphenyldimethyldicarboxylate | 7.5 |
| Transcutol | 400 |
| Polyethyleneglycol 400 | 100 |
| Cremophor ® RH40 (BASF) | 340 |
| Propyleneglycol monocaprylate (NIKKOL) | 180 |
| Captex ® 300 (Abitec) | 180 |

### EXAMPLE 3

### Preparation of a Soft Capsule Containing a Microemulsion Composition

A soft capsule was prepared by the procedure of Example 1 using the following ingredients:

| | Quantity(mg/capsule) |
|---|---|
| Biphenyldimethyldicarboxylate | 3 |
| Transcutol | 96 |
| Polyethyleneglycol 400 | 192 |
| Cremophor ® RH40 (BASF) | 60 |
| Tween ® 20 (ICI) | 20 |
| Ethyl linoleate | 24 |

### EXAMPLE 4

### Preparation of a Soft Capsule Containing a Microemulsion Composition

A soft capsule was prepared by the procedure of Example 1 using the following ingredients:

| | Quantity(mg/capsule) |
|---|---|
| Biphenyldimethyldicarboxylate | 3 |
| Transcutol | 120 |
| Polyethyleneglycol 400 | 112 |
| Cremophor ® RH40 (BASF) | 80 |
| Propyleneglycol monocaprylate (NIKKOL) | 80 |

### EXAMPLE 5

### Preparation of a Soft Capsule Containing a Microemulsion Composition

A soft capsule was prepared by the procedure of Example 1 using the following ingredients:

| | Quantity(mg/capsule) |
|---|---|
| Biphenyldimethyldicarboxylate | 3 |
| Transcutol | 120 |
| Polyethyleneglycol 400 | 112 |
| Cremophor ® RH40 (BASF) | 124 |
| Propyleneglycol monocaprylate (NIKKOL) | 60 |
| Captex ® 300 (Abitec) | 60 |

### EXAMPLE 6

### Preparation of a Soft Capsule Containing a Microemulsion Composition

A soft capsule was prepared by the procedure of Example 1 using the following ingredients:

| | Quantity(mg/capsule) |
|---|---|
| Biphenyldimethyldicarboxylate | 3 |
| Transcutol | 180 |
| Cremophor ® RH40 (BASF) | 152 |
| Propyleneglycol monocaprylate (NIKKOL) | 72 |
| Captex ® 300 (Abitec) | 72 |

### EXAMPLE 7

### Preparation of a Soft Capsule Containing a Microemulsion Composition

A soft capsule was prepared by the procedure of Example 1 using the following ingredients:

| | Quantity(mg/capsule) |
|---|---|
| Biphenyldimethyldicarboxylate | 3 |
| Transcutol | 140 |
| Polyethyleneglycol 400 | 140 |
| Cremophor ® RH40 (BASF) | 60 |
| Tween ® 20 (ICI) | 20 |
| Ethyl linoleate | 24 |
| Imwitor ® 375 (Huls) | 40 |

### EXAMPLE 8

### Preparation of a Soft Capsule Containing a Microemulsion Composition

A soft capsule was prepared by the procedure of Example 1 using the following ingredients:

| | Quantity(mg/capsule) |
|---|---|
| Biphenyldimethyldicarboxylate | 3 |
| Transcutol | 120 |
| Polyethyleneglycol 400 | 112 |
| Cremophor ® RH40 (BASF) | 80 |
| Propyleneglycol monocaprylate (NIKKOL) | 40 |
| Captex ® 300 (Abitec) | 40 |
| Labrafil ® M2125 CS (Gattefosse) | 20 |

### EXAMPLE 9

### Preparation of a Soft Capsule Containing a Microemulsion Composition

A soft capsule was prepared by the procedure of Example 1 using the following ingredients:

| | Quantity(mg/capsule) |
|---|---|
| Biphenyldimethyldicarboxylate | 3 |
| Transcutol | 216 |
| Cremophor ® RH40 (BASF) | 116 |
| Propyleneglycol monocaprylate (NIKKOL) | 72 |
| Captex ® 300 (Abitec) | 72 |

### EXAMPLE 10

### Preparation of a Soft Capsule Containing a Microemulsion Composition

A soft capsule was prepared by the procedure of Example 1 using the following ingredients:

| | Quantity(mg/capsule) |
|---|---|
| Biphenyldimethyldicarboxylate | 3 |
| Transcutol | 120 |
| Polyethyleneglycol 400 | 112 |
| Labrasol ® (GATTEFOSSE) | 80 |
| Propyleneglycol monocaprylate (NIKKOL) | 80 |

### TEST EXAMPLE 1

### Dissolution Test

The capsules prepared in Example 1, 3 and 10, and the commercially available Nissel® tablet (Taerim Pharm.) as a comparative preparation were subjected to a dissolution test in accordance with the dissolution test method described in Korea pharmacopoeia (the paddle method). Aliquots of each solution were taken at regular time intervals and filtered through a 0.45 μm membrane filter. The amount of biphenyldimethyldicarboxylate dissolved in each sample was determined using the following method:

Test apparatus: Erweka DT 80

Test solutions: 900 ml each of pH 1.2 buffer, pH 4.0 buffer, pH 6.8 buffer and distilled water

Temperature of test solutions: 37±0.5° C.

Rotation speed: 100±2 rpm

Sampling time: 5, 10, 15, 30, 45, 60, 90, 120, 150, 180, 240 and 360 min.

Analysis method: liquid chromatography
Column: Inertsil ODS2 (150 mm×4.6 mm)
Mobile phase: 50% acetonitrile
Injection volume: 20 μl
Flow rate: 1.2 ml/min.
Detector: UV 278 nm

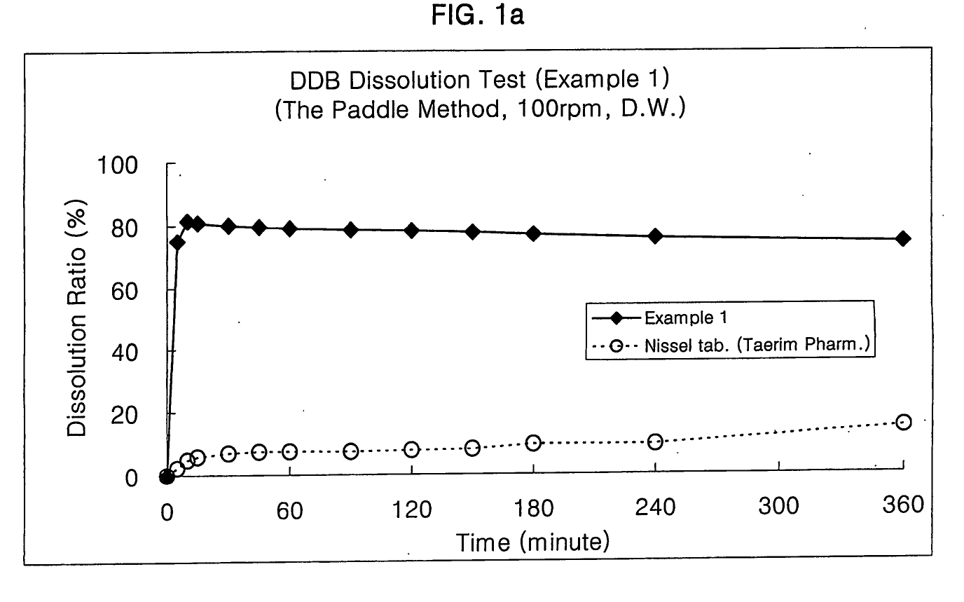

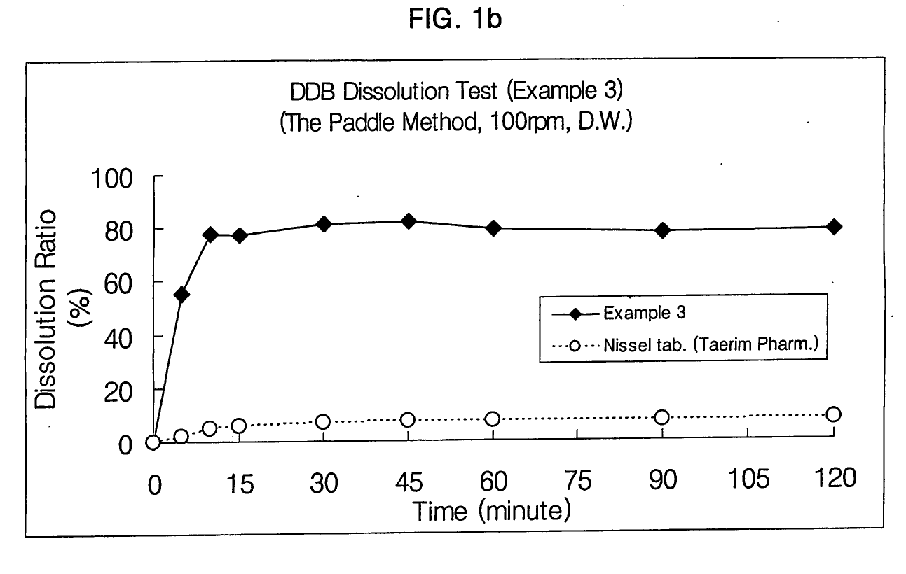

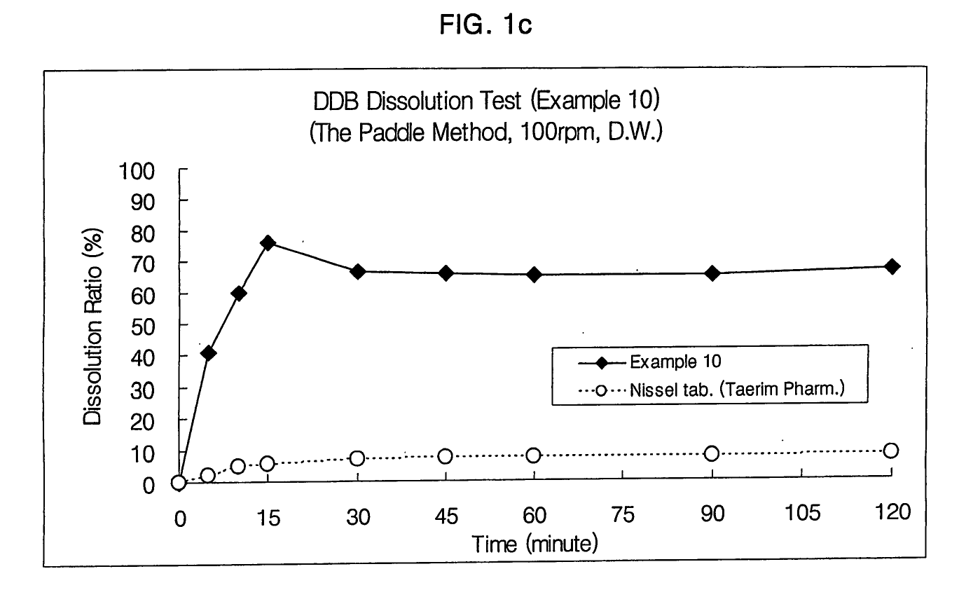

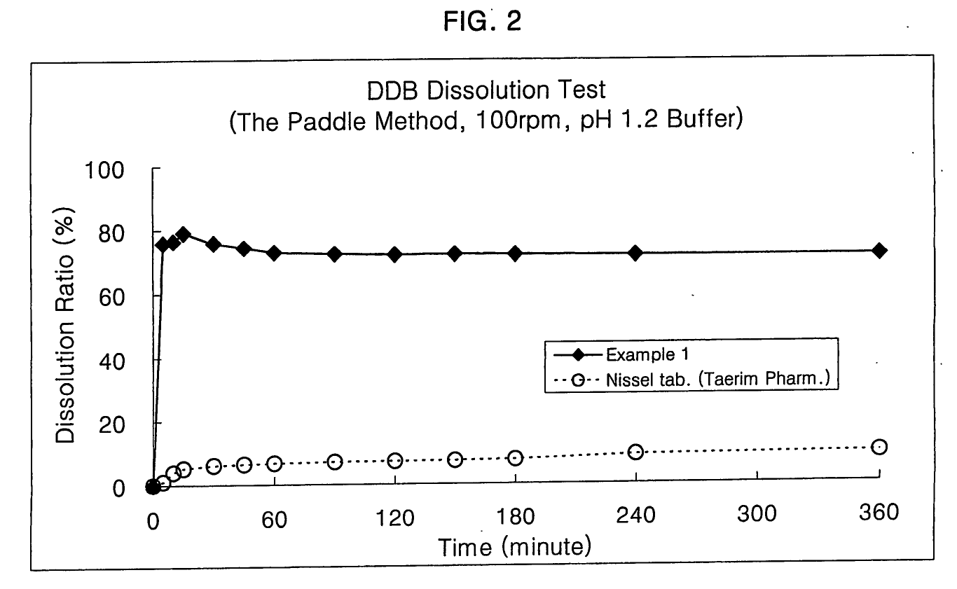

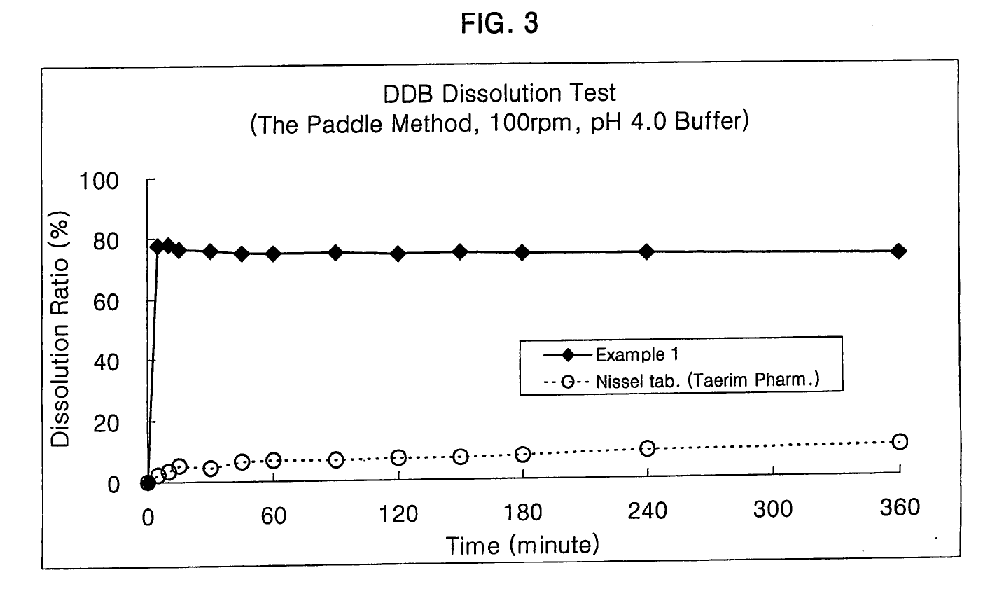

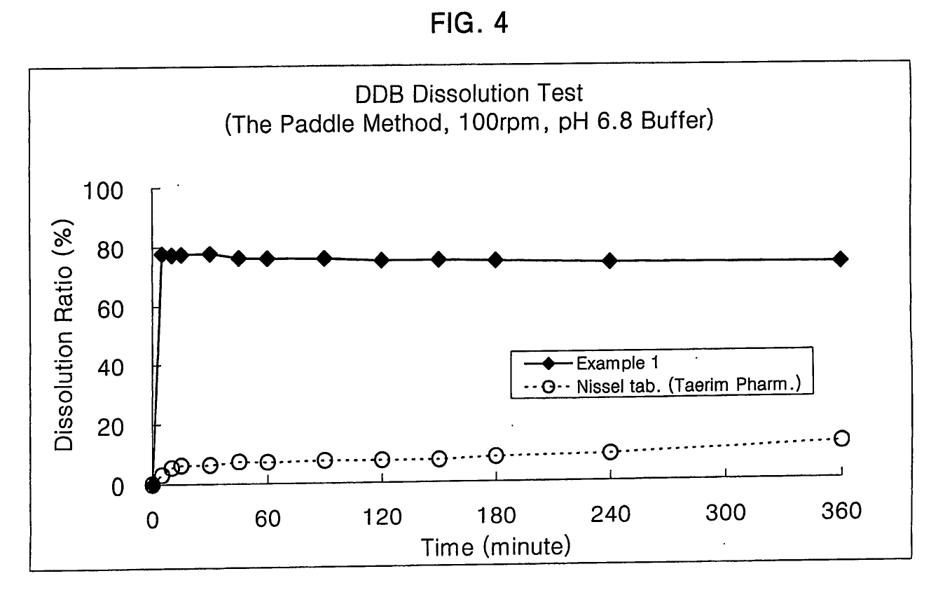

The time-dependent changes in the amount of dissolved biphenyldimethyldicarboxylate are shown in FIGS. 1a to 4 (FIG. 1a: Example 1 in distilled water, FIG. 1b: Example 3 in distilled water, FIG. 1c: Example 10 in distilled water, FIG. 2: Example 1 in pH 1.2 buffer, FIG. 3: Example 1 in pH 4.0 buffer, FIG. 4: Example 1 in pH 6.8).

As shown in FIGS. 1a to 4, the microemulsion compositions of the present invention exhibited higher dissolution rates than the comparative preparation at the various pHs tested.

### TEST EXAMPLE 2

### Analysis of the Emulsified Drug Microparticles

In order to examine whether the preparation of Example 1 would spontaneously emulsify to form microparticles upon contact with an aqueous solution, particle size distribution analysis was carried out, as follows.

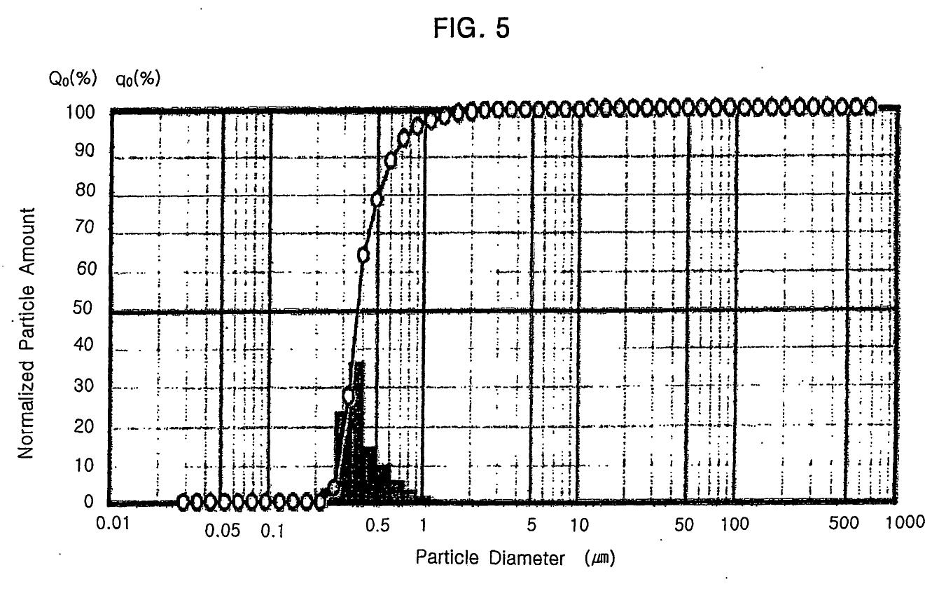

0.1 g of the test preparation was diluted with 10 ml of distilled water, and then, the particle size distribution was determined with a particle analyzer (Shimadzu, SALD-2002 model, Japan). The result is shown in FIG. 5.

As shown in FIG. 5, the inventive microemulsion composition formed emulsified microparticles having an average particle of below 300 nm upon contact with an aqueous solution, to form a microemulsion.

### TEST EXAMPLE 3

### Precipitation Formation Test

In order to examine whether the preparation of Example 1 forms precipitations upon contact with an aqueous solution, 0.1 g each of the preparation of Example 1 and the comparative preparation (G-Cell soft capsule; Guju Pharm., Korean Patent No. 10-201907) was diluted to 10 ml of distilled water, artificial gastric juice or artificial intestinal juice, and then, the formation of precipitating was observed.

The result of the precipitation test is shown in Table 1.

TABLE 1

| | | Artificial gastric | Artificial intestinal |
|---|---|---|---|
| | Distilled water | juice | juice |
| Example 1 | − | − | − |
| Comparative | + | + | + |
| Preparation | | | |

(precipitation: +, no precipitation: −)

As shown in Table 1, the inventive microemulsion preparation does not form precipitating upon contact with an aqueous solution, and therefore, a desired absorption rate and bioavailability improvement can be achieved.

### TEST EXAMPLE 4

### Absorption Test

In order to investigate the bioavailability of the drug contained in the inventive preparation, an in vivo absorption test was carried out as follows by employing the preparation of Example 1 (Experimental preparation) and the commercially available preparation (Nissel®; Taerim Pharm.) as a comparative preparation.

Six 14 to 15-week old male Sprague-Dawley rats (weight: 250 g) were acclimated for more than 4 days while allowing free access to the feed and water. The rats were then put on a 48-hour fast, while they were allowed to free access to water.

The rats were divided into two groups each consisting of three rats, and were orally administered with the experimental and comparative preparations, respectively, in an amount corresponding to 12 mg/kg of biphenyldimethyldicarboxylate. Blood samples were taken from the rats before administration, and 15, 30, 60, 120, 180, 300, 420 min and 24 hours after the administration. 200 μl of methanol were added to 100 μl of plasma, and the mixture was shaken. The mixture was centrifuged at 3,000 rpm for 10 minutes to obtain a supernatant, which was then filtered with a 0.22 μm filter and analyzed by LC-MS, as follows.

Column: Waters MS C18 (2.1×150 mm with guard column)

Mobile phase: 50% methanol

Injection volume: 10 μl

Flow rate: 0.2 ml/min.

Detector: SIR mode m/z: 441.2 (Na adduct)

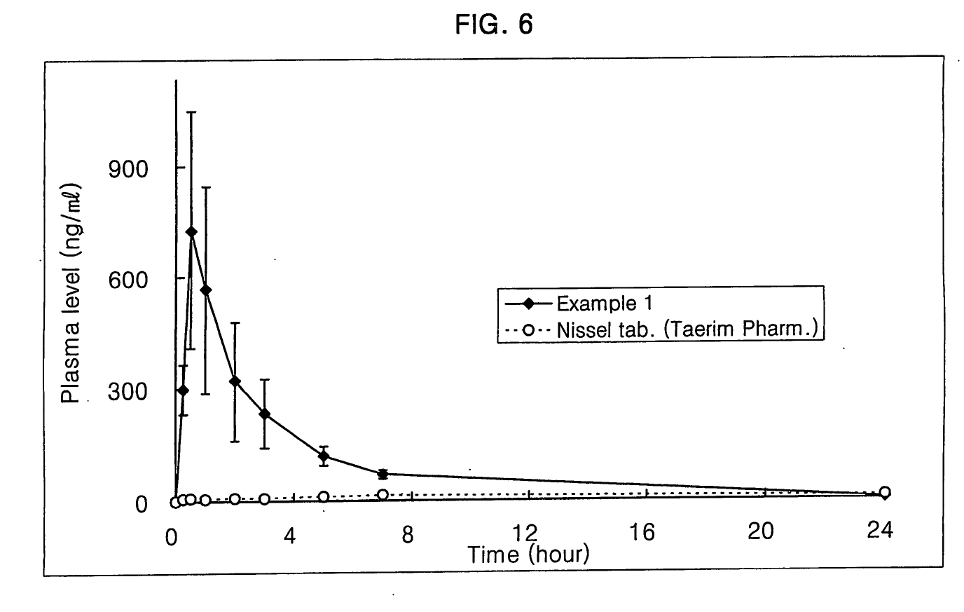

The results are shown in Table 2 and FIG. 6.

TABLE 2

| Preparation | AUC (ng · hr/ml) | Cₘₐₓ (ng/ml) | Tₘₐₓ (hour) |
|---|---|---|---|
| Example 1 | 2383.6 ± 721.7 | 726.0 ± 318.1 | 0.5 ± 0.0 |
| Comparative | 257.3 ± 114.3 | 22.6 ± 7.6 | 2.6 ± 2.2 |
| Preparation | | | |

AUC: Area under the plasma concentration versus time curve integrated for 0 to 24 hours
Cₘₐₓ: Maximum blood concentration
Tₘₐₓ: Time at the maximum blood concentration

As shown in Table 2 and FIG. 6, the bioavailability of the inventive preparation of Example 1 was improved than Nissel® tablet about more than 9 times.

While the invention has been described with respect to the above specific embodiments, it should be recognized that various modifications and changes may be made to the invention by those skilled in the art which also fall within the scope of the invention as defined by the appended claims.

What is claimed is:

1. A microemulsion composition for oral administration of biphenyldimethyldicarboxylate (DDB) comprising biphenyldimethyldicarboxylate, a co-surfactant, a surfactant and an oil.

2. The composition of claim 1, wherein the biphenyldimethyldicarboxylate: co-surfactant: surfactant: oil ratio by weight is in the range of 1:5˜300:1˜300:1˜300.

3. The composition of claim 1, wherein the co-surfactant is selected from the group consisting of transcutol, polyethyleneglycol and a mixture thereof.

4. The composition of claim 1, wherein the surfactant is selected from the group consisting of polyoxyethylene glycolated natural or hydrogenated vegetable oils; polyoxyethylene-sorbitan-fatty acid esters; polyoxyethylene fatty acid esters; polyoxyethylene-polyoxypropylene block copolymers; mono-, di- or mono/di-glycerides; sorbitan fatty acid esters; trans-esterification products of natural vegetable oil triglycerides and polyalkylene polyols; and a mixture thereof.

5. The composition of claim 1, wherein the oil is selected from the group consisting of fatty acid triglycerides; mono-, di- or mono/di-glycerides; esters of fatty acids and monovalent alkanols; propyleneglycol mono- or di-fatty acid esters; squalene and squalane; tocopherol, tocopherol acetate, tocopherol succinate and polyethyleneglycol-1000-tocopherol succinate; and a mixture thereof.

6. The composition of claim 1, which forms microparticles having an average particle of below 300 nm upon contact with an aqueous solution.
